(19) United States
(12) Patent Application Publication
(10) Pub. No.: US 2025/0018200 A1
(43) Pub. Date: Jan. 16, 2025

(54) Computer Implemented Method and System for Protecting a Patient Critical Firmware Function of an Implantable Medical Device

(71) Applicant: BIOTRONIK SE & Co. KG, Berlin (DE)

(72) Inventor: Klaus Schmolinsky, Berlin (DE)

(73) Assignee: BIOTRONIK SE & Co. KG, Berlin (DE)

(21) Appl. No.: 18/708,798

(22) PCT Filed: Nov. 10, 2022
(86) PCT No.: PCT/EP2022/081499

§ 371 Date: May 9, 2024

(30) Foreign Application Priority Data
Nov. 19, 2021 (EP) 21209161.5

(51) Int. Cl. A61N 1/372; A61N 1/02; G16H 40/40

(52) U.S. Cl. CPC A61N 1/37254; A61N 1/025; A61N 1/37264; G16H 40/40

(57) ABSTRACT

A computer implemented method for protecting a patient critical firmware function of an implantable medical device, in particular a pacemaker, a defibrillator and/or a neuro-stimulator, against unintended execution comprising the step of writing the second checksum to a memory area of the implantable medical device from which a checksum is read before execution of the patient critical firmware function of the implantable medical device. Furthermore, the invention relates to a system for protecting a patient critical firmware function of an implantable medical device. In addition, a computer program and a computer-readable data carrier are also provided.

### CROSS-REFERENCE TO RELATED APPLICATIONS

This application is the United States National Phase under 35 U.S.C. § 371 of PCT International Patent Application No. PCT/EP2022/081499, filed on Nov. 10, 2022, which claims the benefit of European Patent Application No. 21209161.5, filed on Nov. 19, 2021, the disclosures of which are hereby incorporated by reference herein in their entireties.

### TECHNICAL FIELD

The present invention relates to a computer implemented method for protecting a patient critical firmware function of an implantable medical device, in particular a pacemaker, a defibrillator and/or a neuro-stimulator, against unintended execution.

Furthermore, the present invention relates to a system for protecting a patient critical firmware function of an implantable medical device, in particular a pacemaker, a defibrillator and/or a neuro-stimulator, against unintended execution.

Patient-critical firmware functions such as Brady_OFF_Mode, Therapy_OFF_State, or similar of implants, e.g., ICDs, S-ICD, IPGs, iLPs, or similar active implants must be protected from unintended execution due to internal firmware errors and/or misuse by cyberattacks.

### BACKGROUND

European Publication No. 3 791 925 A1 discloses a leadless pacemaker comprising at least one fixation element for fixating the leadless pacemaker to cardiac tissue, a communication unit which is electrically connected to said fixation element, so that said fixation element is configured to act as a communication antenna for transmitting signals generated by said communication unit to an external device and/or receiving signals from an external device, and a therapy unit for generating electrical signals to electrically stimulate cardiac tissue, wherein said fixation element is configured to act as an electrode for electrically stimulating cardiac tissue and/or sensing electrical signals of the cardiac tissue.

Currently with such implants, certain firmware functions are protected with appropriate checksums to check for potential code modifications, e.g., by bitflips before execution and thus prevent their execution. However, no distinction is made between regular firmware functions and the above-mentioned patient-critical firmware functions which should be protected by an additional layer of security.

The present disclosure is directed toward overcoming one or more of the above-mentioned problems, though not necessarily limited to embodiments that do.

### SUMMARY

It is therefore an object of the present invention to provide an improved method for protecting a patient critical firmware function of an implantable medical device against unintended execution.

At least the object is solved by a computer implemented method for protecting a patient critical firmware function of an implantable medical device having the features of claim 1.

At least the object is furthermore solved by a system for protecting a patient critical firmware function of an implantable medical device having the features of claim 10.

In addition, the at least the object is solved by a computer program of claim 11 and the computer-readable data carrier of claim 12. Further developments and advantageous embodiments are defined in the dependent claims.

The present invention provides a computer implemented method for protecting a patient critical firmware function of an implantable medical device, in particular a pacemaker, a defibrillator and/or a neuro-stimulator, against unintended execution.

The method comprises receiving a request for execution of a patient critical firmware function of the implantable medical device and verifying that a user associated with the request is authorized to run the patient critical firmware function.

Furthermore, the method comprises, if the user associated with the request is verified as authorized, reading a first checksum from a first memory area of the implantable medical device or providing a first checksum as part of a code area of the patient critical firmware function of the implantable medical device, wherein the first checksum does not match a correct checksum associated with the patient critical firmware function of the implantable medical device.

The method additionally comprises reading a second checksum from a second memory area of the implantable medical device, wherein the second checksum matches the correct checksum associated with the patient critical firmware function of the implantable medical device and writing the second checksum to a third memory area of the implantable medical device from which a checksum is read before execution of the patient critical firmware function of the implantable medical device.

Moreover, the method comprises computing the correct checksum associated with the patient critical firmware function of the implantable medical device and comparing it to the second checksum and, if the second checksum and the correct checksum match, executing the patient critical firmware function of the implantable medical device.

Furthermore, the present invention provides a system for protecting a patient critical firmware function of an implantable medical device, in particular a pacemaker, a defibrillator and/or a neuro-stimulator, against unintended execution.

The system comprises an implantable medical device and a programmer, wherein the implantable medical device is configured to receive a request by the programmer for execution of a patient critical firmware function of the implantable medical device, wherein the implantable medical device is configured to verify that a user associated with the request is authorized to run the patient critical firmware function, wherein the implantable medical device is configured to read a first checksum from a first memory area of the implantable medical device or to provide a first checksum as part of a code area of the patient critical firmware function of the implantable medical device, wherein the first checksum does not match a correct checksum associated with the patient critical firmware function of the implantable medical device.

The implantable medical device is configured to read a second checksum from a second memory area of the implantable medical device, wherein the second checksum matches the correct checksum associated with the patient critical firmware function of the implantable medical device. Furthermore, the implantable medical device is configured to write the second checksum to a third memory area of the implantable medical device from which a checksum is read before execution of the patient critical firmware function of the implantable medical device.

The implantable medical device is configured to compute the correct checksum associated with the patient critical firmware function of the implantable medical device and to compare it to the second checksum, and wherein the implantable medical device is configured to execute the patient critical firmware function of the implantable medical device if the second checksum and the correct checksum match.

Moreover, the present invention provides a computer-readable data carrier containing program code of a computer program for performing the method according to the present invention when the computer program is executed on a computer.

It is an idea of the present invention to provide a patient critical firmware function with a highly secure checksum, which by default is always incorrect.

This reliably prevents the implant firmware from accidentally or intentionally starting a critical firmware function, e.g., programming of OFF mode for IPGs, Therapy_OFF state for ICDs, etc. due to an internal error or cyber attack, e.g., remotely via a programmer or cardiomessenger, said cardiomessenger being an external BIOTRONIK device that forwards messages and/or data sent by the implant to a Home Monitoring Service Center (HMSC) via mobile radio.

E.g., programming of OFF_mode for IPGs, Therapy_OFF state for ICDs etc. hence requires that a checksum check always precedes the execution of these functions.

It is ensured in the code that this checksum is always checked by the firmware immediately before a critical function is executed. If this is not correct, the function is not executed and, e.g., an abusive execution attempt is reported to an internal firmware log book, which can be transmitted to BIOTRONIK, e.g., during a follow-up or by transmission in a technical HMSC message. Thus, e.g., attempted cyber attacks can be captured by said monitoring.

Through appropriate strong authorization, e.g., password entry on the programmer by the physician or authorized user, the incorrect checksum is always replaced by a correct checksum only immediately before the function is used, thus making the execution of the critical function possible and permissible.

According to an aspect of the present invention, the reading of the first checksum from the first memory area of the implantable medical device or the providing of the first checksum as part of a code area of the patient critical firmware function of the implantable medical device, the reading of the second checksum from a second memory area of the implantable medical device, the writing of the second checksum to a third memory area of the implantable medical device from which a checksum is read before execution of the patient critical firmware function of the implantable medical device, the computing of the correct checksum associated with the patient critical firmware function of the implantable medical device, the comparing it to the second checksum and the execution of the patient critical firmware function of the implantable medical device are performed by a non-interruptible compound command. This way, said method steps are a non-interruptible, which results in an additional layer of security.

According to a further aspect of the present invention, during execution of the compound command of the patient critical firmware function of the implantable medical device, the second checksum is overwritten by the first checksum, said first checksum being read from a memory buffer or from a code area of the patient critical firmware function of the implantable medical device. By overwriting the second checksum by the first checksum, execution of the patient critical firmware function is no longer enabled.

According to a further aspect of the present invention, after overwriting the second checksum by the first checksum, the compound command of the patient critical firmware function of the implantable medical device is terminated. Since the execution of the compound command generally only encompasses transmission of a predefined command to enable or disable specified functions of the implantable medical device, the execution time is short. As soon as the predefined command has been executed the second checksum is thus overwritten by the first checksum thus effectively terminating the compound command.

According to a further aspect of the present invention, the second checksum is read from a hardware read-only register of the implantable medical device, and wherein the checksum is a cyclical redundancy check, XOR, modulus or a cryptographic hash, in particular MD5, SHA-1 or SHA-2. This advantageously the provides an effective protection of the patient critical firmware function.

According to a further aspect of the present invention, the patient critical firmware function of the implantable medical device is executed by accessing a graphical user interface of a programmer or an app operating on mobile device, in particular a smartphone or tablet device, said programmer or mobile device being configured to communicate wirelessly with the implantable medical device. The implantable medical device can thus be accessed in a plurality of manners.

According to a further aspect of the present invention, a user authentication procedure comprises a password input or a two-factor authentication comprising a password input and an additional security feature on the programmer or a web-interface configured to control the programmer, and wherein the user session comprises a session ID and a timestamp. This ensures access to the implantable medical device by only authorized users.

According to a further aspect of the present invention, the correct checksum associated with the patient critical firmware function of the implantable medical device is computed and compared to the second checksum within a predefined time span, in particular up to 500 ms, prior to execution of the patient critical firmware function of the implantable medical device. By limiting the time span for conducting the checksum computation, an additional layer of security is provided.

According to a further aspect of the present invention, the second checksum is written at a factory initialization of the implantable device to a predefined memory cell, said memory cell being accessible by running a predefined register code. Accidental execution of the patient critical firmware function is thus effectively prevented due to the fact that said predefined register code is not part of the code for executing the patient critical firmware function.

The herein described features of the computer implemented method for protecting a patient critical firmware function of an implantable medical device are also disclosed for the system for protecting a patient critical firmware function of an implantable medical device and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a flowchart of a computer implemented method for protecting a patient critical firmware function of an implantable medical device according to a preferred embodiment of the present invention; and

FIG. 2 shows a schematic view of a system for protecting a patient critical firmware function of an implantable medical device according to the preferred embodiment of the present invention.

### DETAILED DESCRIPTION

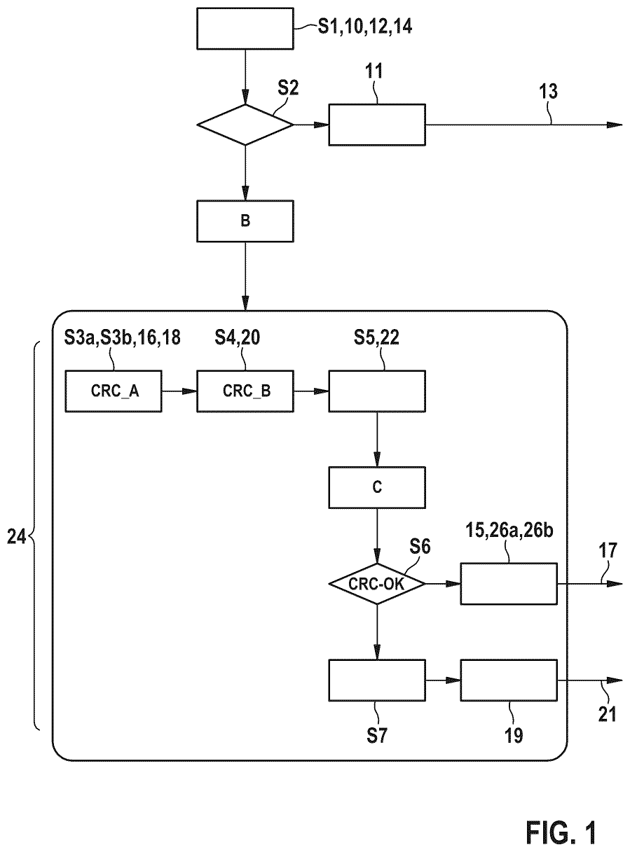

The computer implemented method of FIG. 1 serves to protect a patient critical firmware function 10 of an implantable medical device 12, in particular a pacemaker, a defibrillator and/or a neuro-stimulator, against unintended execution.

The method comprises receiving S1 a request 14 for execution of a patient critical firmware function 10 of the implantable medical device 12 and verifying S2 that a user associated with the request 14 is authorized to run the patient critical firmware function 10.

If the user is confirmed to be authorized, a composite command 24 in step B is started. If the user is not authorized, use of the patient critical firmware function 10 is denied. In addition or optionally an entry in the cyber logbook can be made in step 11. This in turn will cancel the (command) request 14 in step 13.

Furthermore, the method comprises, if the user associated with the request 14 is verified as authorized, reading S3a a first checksum CRC_A from a first memory area 16 of the implantable medical device 12 or providing S3b a first checksum CRC_A as part of a code area 18 of the patient critical firmware function 10 of the implantable medical device 12. The first checksum CRC_A does not match a correct checksum CRC_OK associated with the patient critical firmware function 10 of the implantable medical device 12.

The method additionally comprises reading S4 a second checksum CRC_B from a second memory area 20 of the implantable medical device 12, wherein the second checksum CRC_B matches the correct checksum CRC_OK associated with the patient critical firmware function 10 of the implantable medical device 12.

Moreover, the method comprises writing S5 the second checksum CRC_B to a third memory area 22 of the implantable medical device 12 from which a checksum is read before execution of the patient critical firmware function 10 of the implantable medical device 12. Subsequently, patient critical firmware function 10 is started in step C.

Furthermore, the method comprises computing S6 the correct checksum CRC_OK associated with the patient critical firmware function 10 of the implantable medical device 12 and comparing it to the second checksum CRC_B. If the second checksum CRC_B and the correct checksum CRC_OK match, executing S7 the patient critical firmware function 10 of the implantable medical device 12.

If the second checksum CRC_B and the correct checksum CRC_OK do not match, use of the patient critical firmware function is denied and an entry in the error log is made in step 15. Additionally, the composite command is canceled in step 17.

The steps the reading S3a, S3b, S4, S5, S6 and S7 of the patient critical firmware function 10 of the implantable medical device 12 are performed by a non-interruptible compound command 24.

During execution of the compound command 24 of the patient critical firmware function 10 of the implantable medical device 12, the second checksum CRC_B is overwritten in step 19 by the first checksum CRC_A, said first checksum CRC_A being read from a memory buffer 26a or from a further code area 26b of the patient critical firmware function 10 of the implantable medical device 12.

After overwriting the second checksum CRC_B by the first checksum CRC_A in step 19, the compound command 24 of the patient critical firmware function 10 of the implantable medical device 12 is terminated in step 21.

The second checksum CRC_B is read from a hardware read-only register of the implantable medical device 12, and wherein the second checksum CRC_B is a cyclical redundancy check, XOR, modulus or a cryptographic hash, in particular MD5, SHA-1 or SHA-2.

The patient critical firmware function 10 of the implantable medical device 12 is executed by accessing a graphical user interface of a programmer 30 or an app operating on mobile device 32, in particular a smartphone or tablet device, said programmer 30 or mobile device 32 being configured to communicate wirelessly with the implantable medical device 12.

A user authentication procedure comprises a password input or a two-factor authentication comprising a password input and an additional security feature on the programmer 30 or a web-interface configured to control the programmer 30, and wherein the user session comprises a session ID and a timestamp.

The correct checksum CRC_OK associated with the patient critical firmware function 10 of the implantable medical device 12 is computed and compared to the second checksum CRC_B within a predefined time span, in particular up to 500 ms, prior to execution of the patient critical firmware function 10 of the implantable medical device 12.

The second checksum CRC_B is written at a factory initialization of the implantable medical device 12 to a predefined memory cell, said memory cell being accessible by running a predefined register code.

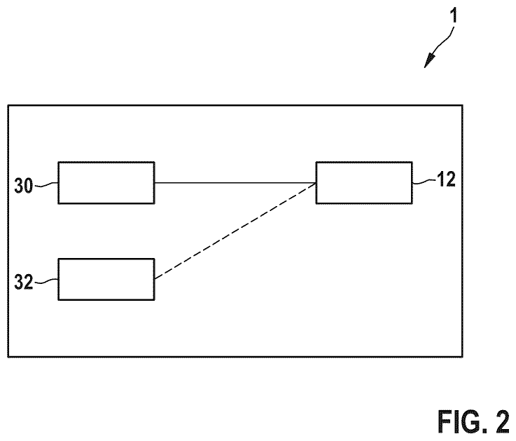

FIG. 2 shows a schematic view of a system for protecting a patient critical firmware function of an implantable medical device according to the preferred embodiment of the present invention.

The system 1 comprises an implantable medical device 12 and a programmer 30. Alternatively, the implantable medical device 12 may be controlled by a mobile device 32 being configured to communicate wirelessly with the implantable medical device 12.

The implantable medical device 12 is configured to receive a request 14 by the programmer 30 for execution of a patient critical firmware function 10 of the implantable medical device 12, wherein the implantable medical device 12 is configured to verify that a user associated with the request 14 is authorized to run the patient critical firmware function 10.

The implantable medical device 12 is configured to read a first checksum CRC_A from a first memory area 16 of the implantable medical device 12 or to provide a first checksum CRC_A as part of a code area 18 of the patient critical firmware function 10 of the implantable medical device 12, wherein the first checksum CRC_A does not match a correct checksum CRC_OK associated with the patient critical firmware function 10 of the implantable medical device 12.

Furthermore, the implantable medical device 12 is configured to read a second checksum CRC_B from a second memory area 20 of the implantable medical device 12, wherein the second checksum CRC_B matches the correct checksum CRC_OK associated with the patient critical firmware function 10 of the implantable medical device 12.

Moreover, the implantable medical device 12 is configured to write the second checksum CRC_B to a third memory area 22 of the implantable medical device 12 from which a checksum is read before execution of the patient critical firmware function 10 of the implantable medical device 12.

The implantable medical device 12 is further configured to compute the correct checksum CRC_OK associated with the patient critical firmware function 10 of the implantable medical device 12 and to compare it to the second checksum CRC_B. The implantable medical device 12 is configured to execute the patient critical firmware function 10 of the implantable medical device 12 if the second checksum CRC_B and the correct checksum CRC_OK match.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

1 system
10 patient critical firmware function
11 method step
12 implantable medical device
13 method step
14 request
15 method step
16 first memory area
17 method step
18 code area
19 method step
20 second memory area
21 method step
22 third memory area
24 compound command
26a memory buffer
26b further code area
30 programmer
32 mobile device
B method step
C method step
CRC_A first checksum
CRC_B second checksum
CRC_OK correct checksum
S1-S7 method steps

1. Computer implemented method for protecting a patient critical firmware function of an implantable medical device, in particular a pacemaker, a defibrillator and/or a neuro-stimulator, against unintended execution, the method comprising the steps of: receiving a request for execution of a patient critical firmware function of the implantable medical device;
verifying that a user associated with the request is authorized to run the patient critical firmware function;
if the user associated with the request is verified as authorized, reading a first checksum from a first memory area of the implantable medical device or providing a first checksum as part of a code area of the patient critical firmware function of the implantable medical device, wherein the first checksum does not match a correct checksum associated with the patient critical firmware function of the implantable medical device;
reading a second checksum from a second memory area of the implantable medical device, wherein the second checksum matches the correct checksum associated with the patient critical firmware function of the implantable medical device;
writing the second checksum to a third memory area of the implantable medical device from which a checksum is read before execution of the patient critical firmware function of the implantable medical device;
computing the correct checksum associated with the patient critical firmware function of the implantable medical device and comparing it to the second checksum; and
if the second checksum and the correct checksum match, executing the patient critical firmware function of the implantable medical device.

2. Computer implemented method of claim 1, wherein the reading of the first checksum (CRC_A) from the first memory area of the implantable medical device or the providing of the first checksum as part of the code area of the patient critical firmware function of the implantable medical device, the reading of the second checksum from the second memory area of the implantable medical device, the writing of the second checksum to the third memory area of the implantable medical device from which the checksum is read before execution of the patient critical firmware function of the implantable medical device, the computing of the correct checksum associated with the patient critical firmware function of the implantable medical device, the comparing it to the second checksum and the execution of the patient critical firmware function of the implantable medical device are performed by a non-interruptible compound command.

3. Computer implemented method of claim 2, wherein during execution of the compound command of the patient critical firmware function of the implantable medical device, the second checksum is overwritten by the first checksum, said first checksum being read from a memory buffer or from a further code area of the patient critical firmware function of the implantable medical device.

4. Computer implemented method of claim 3, wherein after overwriting the second checksum by the first checksum, the compound command of the patient critical firmware function of the implantable medical device is terminated.

5. Computer implemented method of claim 1, wherein the second checksum is read from a hardware read-only register of the implantable medical device, and wherein the second checksum is a cyclical redundancy check, XOR, modulus or a cryptographic hash, in particular MD5, SHA-1 or SHA-2.

6. Computer implemented method of claim 1, wherein the patient critical firmware function of the implantable medical device is executed by accessing a graphical user interface of a programmer or an app operating on mobile device, in particular a smartphone or tablet device, said programmer or mobile device being configured to communicate wirelessly with the implantable medical device.

7. Computer implemented method of claim 6, wherein a user authentication procedure comprises a password input or a two-factor authentication comprising a password input and an additional security feature on the programmer or a web-interface configured to control the programmer, and wherein the user session comprises a session ID and a timestamp.

8. Computer implemented method of claim 1, wherein the correct checksum associated with the patient critical firmware function of the implantable medical device is computed and compared to the second checksum within a predefined time span, in particular up to 500 ms, prior to execution of the patient critical firmware function of the implantable medical device.

9. Computer implemented method of claim 1, wherein the second checksum is written at a factory initialization of the implantable medical device to a predefined memory cell, said memory cell being accessible by running a predefined register code.

10. System for protecting a patient critical firmware function of an implantable medical device, in particular a pacemaker, a defibrillator and/or a neuro-stimulator, against unintended execution, the system comprising:
an implantable medical device and a programmer, wherein the implantable medical device is configured to receive a request by the programmer for execution of a patient critical firmware function of the implantable medical device, wherein the implantable medical device is configured to verify that a user associated with the request is authorized to run the patient critical firmware function, wherein the implantable medical device is configured to read a first checksum from a first memory area of the implantable medical device or to provide a first checksum as part of a code area of the patient critical firmware function of the implantable medical device, wherein the first checksum does not match a correct checksum associated with the patient critical firmware function of the implantable medical device, wherein the implantable medical device is configured to read a second checksum from a second memory area of the implantable medical device, wherein the second checksum matches the correct checksum associated with the patient critical firmware function of the implantable medical device, wherein the implantable medical device is configured to write the second checksum to a third memory area of the implantable medical device from which a checksum is read before execution of the patient critical firmware function of the implantable medical device, wherein the implantable medical device is configured to compute the correct checksum associated with the patient critical firmware function of the implantable medical device and to compare it to the second checksum, and wherein the implantable medical device is configured to execute the patient critical firmware function of the implantable medical device if the second checksum and the correct checksum match.

11. Computer program with program code to perform the method of claim 1 when the computer program is executed on a computer.

12. Computer-readable data carrier containing program code of a computer program for performing the method of claim 1 when the computer program is executed on a computer.
